(19) United States
(12) Patent Application Publication
(10) Pub. No.: US 2026/0307032 A1
(43) Pub. Date: Oct. 8, 2026

(54) REACTOR EVACUATION METHOD AND SYSTEM FOR DEHYDROGENATION REACTOR

(71) Applicant: SABIC GLOBAL TECHNOLOGIES B.V., Bergen op Zoom (NL)

(72) Inventors: Sethuraman BALASUBRAMANIYAN, Bangalore (IN); Cesar Enrique RAMIREZ, Doha (QA); Muhammad Imran NADEEM, Amsterdam (NL); Srikant Vasant BADGANDI, Al-Jubail (SA)

(21) Appl. No.: 19/482,921

(22) PCT Filed: May 13, 2024
(86) PCT No.: PCT/EP2024/063059

§ 371 Date: Nov. 10, 2025

(30) Foreign Application Priority Data
May 19, 2023 (EP) 23174266.9

(51) Int. Cl. B01J 8/00; B01J 8/02

(52) U.S. Cl. CPC B01J 8/001; B01J 8/0278; B01J 8/0285; B01J 2208/00362; B01J 2208/00539; B01J 2208/00637

(57) ABSTRACT

The present disclosure provides a method for evacuating a high temperature reactor requiring vacuum pressure during one or more steps of the reaction process, including directing a reduction gas stream from the reactor to a quench system in fluid communication with the reactor, the quench system comprising one or more enclosures wherein the reduction gas stream directly contacts a liquid cooling medium, wherein the liquid cooling medium is water; withdrawing a cooled gas stream from the quench system; and directing the cooled gas stream to a vacuum pump in fluid communication with the quench system and the reactor to reduce the pressure within the reactor to a vacuum pressure. A system for evacuating a high temperature reactor is also provided, including a high temperature reactor combined with a quench system and a vacuum pump as described above.

### TECHNOLOGICAL FIELD

The present disclosure relates to methods and systems for evacuating a high temperature reactor, as well as methods and systems for dehydrogenation of a hydrocarbon.

### BACKGROUND

Fixed bed dehydrogenation units are used for production of olefins and/or alkynes from alkanes and/or olefins. Generally, a fixed bed dehydrogenation unit comprises three or more parallel fixed bed reactors and a catalyst regeneration system. When the fixed bed dehydrogenation unit is in operation, one or more reactors are on line (in dehydrogenation mode), and one or more fixed bed reactors are in regeneration mode.

A fixed bed reactor in dehydrogenation mode first dehydrogenates the hydrocarbon feed for a period of time. Then, the fixed bed reactor is purged with steam. In a subsequent regeneration mode, heated air is blown through to decoke the catalyst disposed in the fixed bed reactor. The reactor is in turn evacuated and the catalyst in the reactor undergoes reduction. After catalyst reduction, the reactor is placed back on line for dehydrogenation reaction. The same sequence is repeated automatically for each fixed bed reactor using a programmable logic controller (PLC) to ensure continuous production of the entire dehydrogenation unit.

The fixed bed dehydrogenation reactors operate under vacuum during one or more steps of the dehydrogenation process and especially during dehydrogenation and reduction steps. While the vacuum for the dehydrogenation step is directly created by a process gas compressor located downstream (post a series of gas cooling unit operations), the vacuum for the reduction step is typically created by steam ejectors using steam as a motive fluid. This is due to the fact that reduction products are typical non-process/waste gases. Typically, in large dehydrogenation units, about 20-30 t/hr of steam at 12-13 bar abs is vented through this ejector. Preceding the reduction step, the regeneration air products that remain in the reactor system need to be completely evacuated. The evacuation is done cyclically because air regeneration of the reactor occurs under nominal pressure, thus requiring evacuation of the reactor during each production cycle. Since the steam passing through the ejector is vented to atmosphere, use of steam as a motive fluid in such large amounts presents a significant cost. There remains a need in the art to decrease the cost and improve the efficiency of operating dehydrogenation reactors.

### BRIEF SUMMARY

Example implementations of the present disclosure are directed to processes and systems for dehydrogenation of a hydrocarbon, as well as more generally, to methods and systems for evacuating any high temperature reactor requiring vacuum pressure during one or more steps of the reaction process. In certain embodiments, the present disclosure provides a method for evacuating a high temperature reactor, which includes directing a reduction gas stream from the reactor to a quench system in fluid communication with the reactor, the quench system comprising one or more enclosures wherein the reduction gas stream directly contacts a liquid cooling medium, wherein the liquid cooling medium is water; withdrawing a cooled gas stream from the quench system; and directing the cooled gas stream to a vacuum pump in fluid communication with the quench system and the reactor to reduce the pressure within the reactor to a vacuum pressure. In this manner, conventional use of an ejector that vents steam to atmosphere in order to evacuate the reactor is avoided, which can improve energy efficiency of the process and reduce cost associated with steam production.

Dehydrogenation systems according to the present disclosure include a dehydrogenation reactor comprising a catalyst bed in fluid communication with a hydrocarbon feed source; a regeneration air source in fluid communication with the dehydrogenation reactor; a reducing gas source in fluid communication with the dehydrogenation reactor; and a reactor evacuation system in fluid communication with the dehydrogenation reactor, such as the reactor evacuation system disclosed herein.

The present disclosure includes, without limitation, the following embodiments.

Embodiment 1: A method of evacuating a high temperature reactor requiring vacuum pressure during one or more process steps (such as during dehydrogenation and/or reduction steps), comprising directing a reduction gas stream from the reactor to a quench system in fluid communication with the reactor, the quench system comprising one or more enclosures wherein the reduction gas stream directly contacts a liquid cooling medium, wherein the liquid cooling medium is water; withdrawing a cooled gas stream from the quench system; and directing the cooled gas stream to a vacuum pump in fluid communication with the quench system and the reactor to reduce the pressure within the reactor to a vacuum pressure.

Embodiment 2: The method of Embodiment 1, wherein the quench system comprises: a pipe in fluid communication with the reactor and equipped with one or more nozzles positioned to spray cooling medium into direct contact with the reduction gas stream within the pipe; and/or a vessel in fluid communication with the reactor and equipped with one or more nozzles positioned to spray cooling medium into direct contact with the reduction gas stream within the vessel, the vessel comprising a gas exit for gaseous effluent and a cooling medium exit for liquid cooling medium effluent; and/or a liquid-cooled heat exchanger in fluid communication with the reactor; and/or any combination of the above.

Embodiment 3: The method of Embodiment 1 or 2, further comprising venting the cooled gas stream from the vacuum pump to atmosphere.

Embodiment 4: The method of any one of Embodiments 1 to 3, wherein the vacuum pump is a dry vacuum pump or a liquid ring vacuum pump.

Embodiment 5: The method of any one of Embodiments 1 to 4, wherein the temperature of the cooled gas stream directed to the vacuum pump is about 150° C. or less, such as about 25° C. to about 120° C. or about 30° C. to about 80° C.

Embodiment 6: The method of any one of Embodiments 1 to 5, wherein the reactor is evacuated to a vacuum pressure of about 0.6 bara or less, such as about 0.2 to about 0.6 bara.

Embodiment 7: The method of any one of Embodiments 1 to 6, wherein the reduction gas stream from the reactor directed to the quench system has a temperature of about 400° C. to about 800° C., such as about 500° C. to about 750° C.

Embodiment 8: The method of any one of Embodiments 1 to 7, wherein the reactor is a dehydrogenation reactor.

Embodiment 9: A system for evacuating a high temperature reactor requiring vacuum pressure during one or more process steps, comprising: a high temperature reactor; a quench system in fluid communication with the reactor and positioned to receive a reduction gas stream from the reactor, the quench system comprising one or more enclosures wherein the reduction gas stream directly contacts a liquid cooling medium, wherein the liquid cooling medium is water; a source of liquid cooling medium in fluid communication with the quench system; and a vacuum pump in fluid communication with the quench system and the reactor and positioned to receive a cooled gas stream from the quench system, the vacuum pump adapted to reduce the pressure within the reactor to a vacuum pressure.

Embodiment 10: The system of Embodiment 9, wherein the quench system comprises: a pipe in fluid communication with the reactor and equipped with one or more nozzles positioned to spray cooling medium into direct contact with the reduction gas stream within the pipe; and/or a vessel in fluid communication with the reactor and equipped with one or more nozzles positioned to spray cooling medium into direct contact with the reduction gas stream within the vessel, the vessel comprising a gas exit for gaseous effluent and a cooling medium exit for liquid cooling medium effluent; and/or a liquid-cooled heat exchanger in fluid communication with the reactor; and/or any combination of the above.

Embodiment 11: The system of Embodiments 9 or 10, wherein the vessel is a vertical quench column comprising a demister proximal to a top of the column, a plurality of spray nozzles adapted for directing a cooling medium spray toward a bottom of the column, either packing or a plurality of trays between the spray nozzles and the bottom of the column, and a gas inlet proximal to the bottom of the column, wherein the gas exit is above the demister.

Embodiment 12: The system of any one of Embodiments 9 to 11, wherein the vacuum pump is vented to atmosphere for release of the cooled gas stream.

Embodiment 13: The system of any one of Embodiments 9 to 12, wherein the vacuum pump is a dry vacuum pump or a liquid ring vacuum pump.

Embodiment 14: The system of any one of Embodiments 9 to 13, wherein the vacuum pump is adapted for evacuating the reactor to a vacuum pressure of about 0.6 bara or less, such as about 0.2 to about 0.6 bara.

Embodiment 15: The system of any one of Embodiments 9 to 14, wherein the reactor is a dehydrogenation reactor.

Embodiment 16: A method of operating a dehydrogenation reactor requiring vacuum pressure during one or more steps of the dehydrogenation process to remove exhaust gases from the reactor to be released to atmosphere, comprising: feeding a hydrocarbon to a reactor comprising a catalyst bed and configured to dehydrogenate the hydrocarbon (e.g., to produce an olefin) under vacuum pressure; optionally purging the reactor to remove remaining hydrocarbon; regenerating the catalyst bed with air from a regeneration air source; evacuating the reactor to induce vacuum pressure, and feeding a reducing gas to the reactor to reduce the catalyst after evacuating the reactor, wherein the evacuating method comprises the method of any one of Embodiments 1 to 8.

Embodiment 17: The method of Embodiment 16, wherein the hydrocarbon is selected from the group consisting of propane, isobutane, pentane, isopentane, n-butane, 1-butene, and combinations thereof.

Embodiment 18: The method of any one of Embodiments 16 or 17, wherein purging the reactor comprises passing steam through the reactor.

Embodiment 19: A system for dehydrogenation of a hydrocarbon, comprising: a dehydrogenation reactor comprising a catalyst bed in fluid communication with a hydrocarbon feed source; a regeneration air source in fluid communication with the dehydrogenation reactor; a reducing gas source in fluid communication with the dehydrogenation reactor, and a reactor evacuation system as set forth in any one of Embodiments 9 to 15.

Embodiment 20: The system of Embodiment 19, further comprising a purge gas source in fluid communication with the dehydrogenation reactor, such as a steam source.

Embodiment 21: The system of Embodiment 19 or 20, wherein the hydrocarbon feed source provide a hydrocarbon selected from the group consisting of propane, isobutane, pentane, isopentane, n-butane, 1-butene, and combinations thereof.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURES

Having thus described aspects of the disclosure in the foregoing general terms, reference will now be made to the accompanying figures, which are not necessarily drawn to scale, and wherein:

FIG. 1 is a schematic representation of a dehydrogenation reactor system according to an example implementation of the present disclosure;

FIG. 2 is a schematic representation of an example embodiment of a single dehydrogenation reactor showing the reactor evacuation system according to an example implementation of the present disclosure;

FIG. 3 graphically illustrates the manner in which water temperature in a liquid ring vacuum pump varies with vapor pressure of water;

FIG. 4 is a schematic representation of an example embodiment of a quench system according to an example implementation of the present disclosure; and

FIG. 5 graphically illustrates the energy savings of an example implementation of the present disclosure in comparison to a conventional steam jet ejector system.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combination” is inclusive of blends, mixtures, alloys, reaction products, and the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form.

The present disclosure relates to a method and system for evacuating a high temperature reactor requiring vacuum pressure during a reaction, and can be applied to any high temperature reaction process that requires reactor evacuation to vacuum pressure. Example processes include various dehydrogenation processes such as CATOFIN® dehydrogenation processes and ODH-E (Oxidative Dehydrogenation of Ethane) processes, such as EDHOX™ technology available from Linde Engineering, as well as other redox oxidative dehydrogenation (redox-ODH) reactions, such as those used for production of styrene as set forth in Zhu, X., Gao, Y., Wang, X. et al. A tailored multi-functional catalyst for ultra-efficient styrene production under a cyclic redox scheme. Nat Commun 12, 1329 (2021). For purposes of illustration only, use of the evacuation system and method of the disclosure is described herein in the context of a dehydrogenation reaction used to convert alkanes to alkenes.

### Dehydrogenation Process and System

In one embodiment, the present disclosure relates to improvements in reactor evacuation for a dehydrogenation process used to convert alkanes to alkenes over, for example, a chromium-alumina catalyst. The dehydrogenation process takes place in fixed bed reactors that operate on a cyclic basis to permit continuous flow of the major process streams. In one cycle, hydrocarbon vapors are dehydrogenated and the reactor is then purged with steam and blown with air to burn off coke. These steps are followed by an evacuation and reduction and then another cycle starts.

In a typical fixed bed dehydrogenation process, an aliphatic hydrocarbon (e.g., propane, isobutane, n-butane, 1-butene, or isopentane) passes through a dehydrogenation catalyst bed and is dehydrogenated to a complementary olefin. The olefin is then flushed from the catalyst bed, the catalyst is regenerated and reduced, and the cycle is repeated. The product (dehydrogenated hydrocarbon) of the fixed bed dehydrogenation unit may comprise, for example, propylene, isobutylene, pentene, isoprene, butadiene, or combinations thereof. The dehydrogenation reactions may include reactions (i) and/or (ii) as follows, where “n” in reactions (i) and (ii) is the number of carbon atoms in a hydrocarbon molecule, and “n” is less than 5:

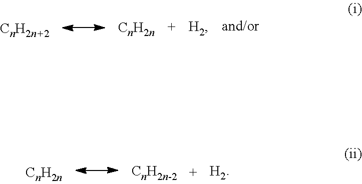

This process can be run as an adiabatic, cyclic process. Each cycle includes a catalyst reduction step and a dehydrogenation step, and typically further includes a step to purge the remaining hydrocarbon from the reactor, and finally a regeneration step with air. Following this, the cycle begins again with the catalyst reduction step.

The reactors in dehydrogenation processes operate under vacuum during various steps of the reaction process, such as during dehydrogenation and during catalyst reduction. The evacuation of the reactors is accomplished using a quench system in combination with a vacuum pump as described in greater detail below. The final reactor pressure after evacuation is typically about 0.6 bar or less, such as about 0.2 to about 0.6 bar (Absolute).

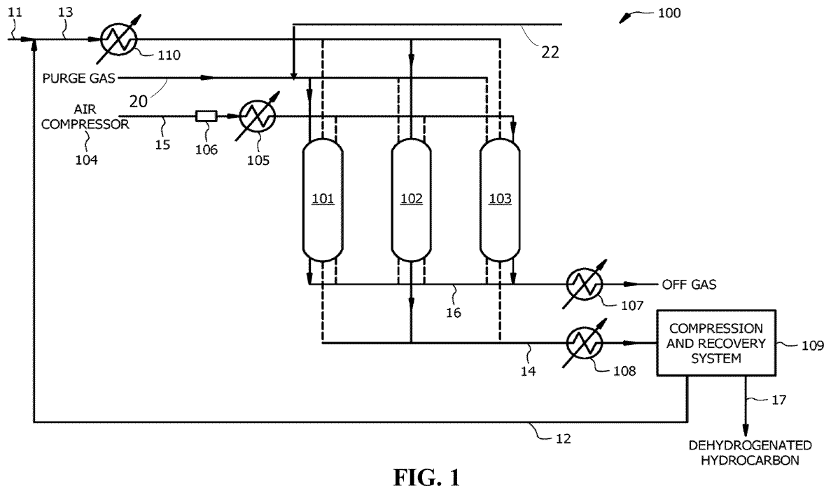

With reference to FIG. 1, a process schematic diagram for an example implementation of a fixed bed dehydrogenation unit 100 is shown, with different reactors at different points in the process cycle. The fixed bed dehydrogenation unit 100 may include fixed bed reactor 101 in purge mode, fixed bed reactor 102 in dehydrogenation mode, and fixed bed reactor 103 in regeneration mode. Each of the fixed bed reactors comprises a catalyst bed. The catalyst may include Cr/Al (chromium oxide over alumina), Sn—Pt/Al (tin-platinum over alumina), or combinations thereof.

The inlet of fixed bed reactor 102 in dehydrogenation mode may be connected to heater 110 that is configured to heat a hydrocarbon feed to a reaction temperature, and the outlet of fixed bed reactor 102 in dehydrogenation mode may be connected to heat exchanger 108 to cool down the effluent from fixed bed reactor 102 in dehydrogenation mode. The combined hydrocarbon stream 13 from hydrocarbon feed stream 11 and recycled hydrocarbon stream 12 may be vaporized and heated to a reaction temperature by heater 110. The reaction temperature is typically about 540° C. to about 750° C. The reaction pressure may be in a range of about 0.2 to about 1.2 bara, such as about 0.2 to about 0.6 bara.

Fixed bed dehydrogenation unit 100 may further include a regeneration air system comprising air compressor 104 configured to blow air into fixed bed reactor 103 in regeneration mode, regeneration air heater 105 configured to heat the air from air compressor 104, fuel injector 106 configured to inject fuel gas into fixed bed reactor 103 in regeneration mode, and heat exchanger 107 configured to cool down the effluents from fixed bed reactor 103 in regeneration mode and fixed bed reactor 101 in purge mode. The effluent from the reactor in purge mode may be configured to be cooled in either of heat exchanger 107 or 108 with a suitable condensate recovery system. Fuel injector 106 may be disposed between air compressor 104 and air heater 105. Stream 16 leaving fixed bed reactor 103 in regeneration mode may be used for generating steam via heat exchanger 107. The regenerating conditions can include a regenerating pressure of about 0.1 to about 10 bar. The regenerating conditions can include a regenerating period that may be in a range of about 7 to about 18 minutes.

The fixed bed dehydrogenation unit 100 may further include a compression and recovery system 109 to recover and purify a dehydrogenated hydrocarbon obtained from fixed bed reactor 102 in dehydrogenation mode. Specifically, effluent stream 14 from fixed bed reactor 102 in dehydrogenation mode may be cooled, recovered, and purified through recovery system 109. Purified dehydrogenated hydrocarbon may flow in stream 17. Recovered unreacted hydrocarbon may be recycled back to combined hydrocarbon stream 13 via recycled hydrocarbon stream 12.

The fixed bed dehydrogenation unit 100 may further include a purge gas source 20 (e.g., steam) in fluid communication with each reactor for use in the purge step and a reducing gas source 22 (e.g., hydrogen) in fluid communication with each reactor to reduce the catalyst.

Process sequence can be controlled, for example, using programmable logic controllers. See, for example, the programmable logic controllers set forth in U.S. Pat. No. 11,370,729 to Ansari et al. and US Pat. Publ. No. 2022/0055002 to Bodas et al, which are incorporated by reference herein in their entirety.

### Evacuation System for Dehydrogenation Reactor

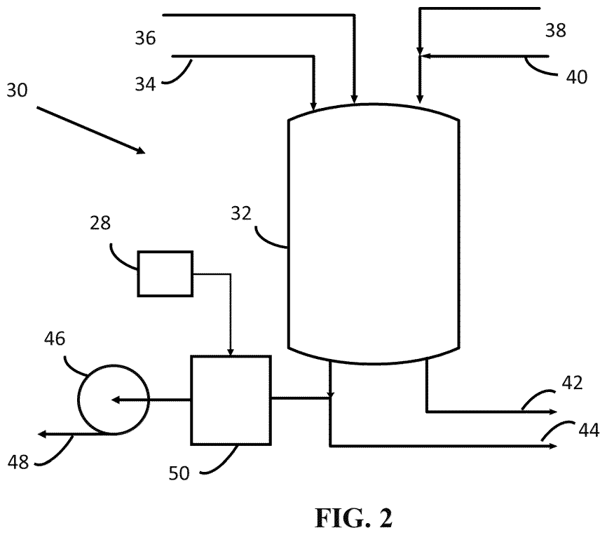

According to the present disclosure, a quench system in combination with a vacuum pump is used for reactor evacuation. An example implementation of a dehydrogenation reactor system 30 is shown in FIG. 2. For the sake of simplicity, a single reactor is illustrated in FIG. 2, but as shown in FIG. 1, dehydrogenation units typically include multiple reactors operated in parallel. The system 30 includes a reactor 32 in fluid communication with a hydrocarbon feed 34, a regeneration air feed 36, a purge gas feed 38, and a reducing gas feed 40. The reactor 32 has a hydrocarbon effluent stream 42 and an off-gas effluent stream 44.

Additionally, as show in FIG. 2, the reactor 32 is also in fluid communication with a quench system 50 and a vacuum pump 46, which are adapted to evacuate the reactor 32 to vacuum pressure. Unlike conventional systems using an ejector receiving steam as a motive gas, the present disclosure utilizes a vacuum pump 46 to evacuate the reactor 32. The quench system 50, explained more fully below, cools the reduction gas received from the reactor 32 before the gas is received by the vacuum pump 46 and ultimately vented as an effluent stream 48. By replacing the need for steam as a motive gas, venting of large amounts of steam to atmosphere is avoided, which increases the energy efficiency of the overall dehydrogenation process and reduces cost associated with steam production.

The temperature of the reduction gas stream from the reactor 32 upstream of the quench system 50 can vary, but is typically within the range of about 400° C. to about 800° C., such as about 500° C. to about 750° C. The cooled gas stream directed to the vacuum pump 46 can vary, but is typically about 150° C. or less, such as about 25° C. to about 120° C. or about 30° C. to about 80° C.

The proposed quench system 50 provides a direct contact reduction gas quenching arrangement comprising at least one enclosure (e.g., a vessel or pipe) where a liquid cooling medium is in direct contact with the reduction gas leaving the reactor 32. The quench system 50 can also include indirect cooling of the reduction gas through, for example, the presence of one or more heat exchangers within the quench system and/or through jacketed pipes or vessels that provide cooling water circulation through the jacket in addition to the direct contact with a liquid cooling medium. As shown, a source of liquid cooling medium 28 (water) is in fluid communication with the quench system, and can be used, for example, as the source of liquid cooling medium for both direct and indirect cooling of the reduction gas provided in the quench system 50.

Example embodiments of a direct contact cooling enclosure include one or more pipes equipped with one or more nozzles for spraying cooling into direct contact with the reduction gas stream within the pipe, and one or more column vessels with an internal nozzle spray system. Such vessels can have a horizontal or vertical orientation, with either a counter-current or co-current flow configuration, and fitted with a nozzle injection system to inject water as liquid cooling medium at any temperature below its boiling point. The quench vessels can have a wet-wall or dry wall configuration, optionally with integrated water separation/knockout. A suitable control scheme can be used to ensure, for example, that the gas temperature leaving the quench system 50 is controlled in part by the water supply rate. Typically, the quench system is adapted for cooling the reduction gas from high temperatures (e.g., ranging from 200-1200° C.) to its saturated temperatures at vacuum pressures.

The cooled gas is drawn into a mechanical vacuum pump 46 where work is done to drive the gas to exit conditions (typically atmospheric). The mechanical vacuum pump can be liquid ring or dry vacuum type driven, for example, by a variable speed electric motor. A suitable control scheme can used to ensure that the variable speed drive caters to pressure change dynamics. The proposed quench scheme overcomes the main limitations of mechanical vacuum pumps for high temperature applications by upstream application of integrated direct contact reduction gas cooling. In certain embodiments, the quench scheme provides superior energy efficiency via use of an electrically driven pump instead of a steam jet ejection system. The quench scheme abates CO₂ emissions directly associated with motive steam production and prevents process water losses to environment associated with ejection.

Example vacuum pumps suitable for use in the system and method disclosed herein include those made by Gardner Denver Nash, LLC and Edwards Ltd. Example vacuum pumps can be dry, such as rotary vane pumps or dry screw vacuum pumps, or liquid ring vacuum pumps.

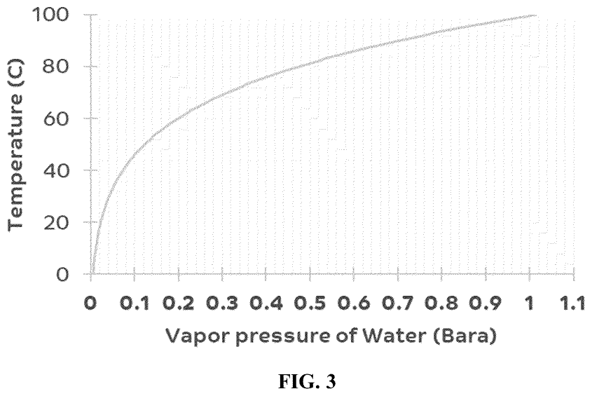

In one embodiment, the vacuum pump is a liquid ring vacuum pump, which typically consists of a rotating vaned impeller eccentrically located within a cylindrical casing. A liquid (typically water or oil) is fed into the pump and forms a moving cylindrical ring against the inside of the casing as the impeller turns. This forms a series of seals in the spaces between the impeller vanes, which form compression chambers. In operation, the gas from the quench system 50 is drawn into the pump through an inlet port, trapped in the compression chambers formed within the liquid ring, and compressed before being discharged. For liquid ring vacuum pumps, the temperature of water as ring medium dictates the minimum achievable vacuum pressure based on vapor pressures of water. An example graph of liquid ring water temperature versus vapor pressure of water is shown in FIG. 3. As shown, lower temperatures of the water in the liquid ring result in lower achievable vapor pressure.

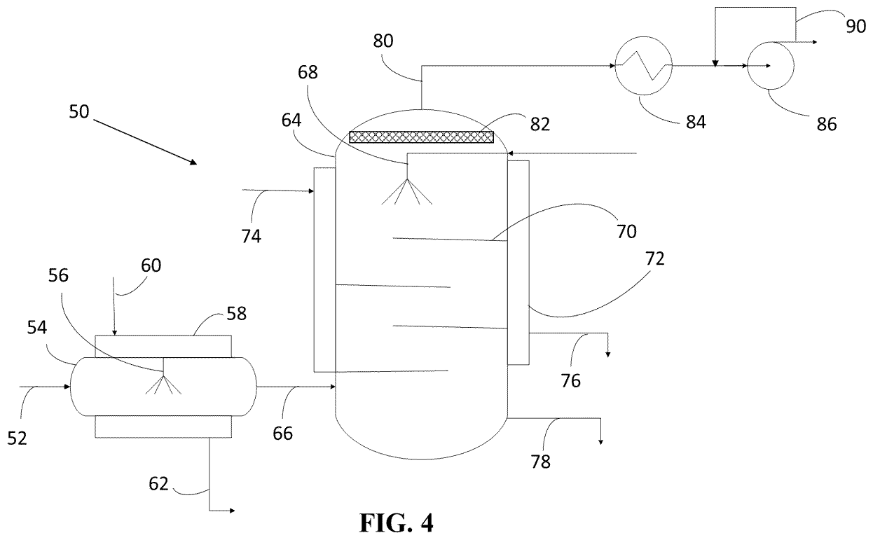

An example implementation of a quench system 50 is shown in FIG. 4. The illustrates embodiment is merely provided as an example system configuration, and can be modified into other configurations without departing from the present disclosure. For example, the order of the unit operations, the configuration of the quench vessel in terms of flow direction and orientation, and the location of any optional heat exchangers can be changed. In the illustrated embodiment, reduction gas stream 52 is received from a reactor (e.g., reactor 32 from FIG. 2) and fed into a pipe 54 equipped with one or more nozzles 56 that spray a liquid cooling medium, such as water, into direct contact with the reduction gas within the pipe. As shown, the pipe 54 is optionally equipped with a cooling water jacket 58 that receives a cooling water feed 60 and produces a cooling water effluent 62. In this manner, the pipe 54 can simultaneously provide both direct and indirect contact with a cooling medium.

The gas effluent 66 from the pipe 54 can enter a quench vessel 64. In the illustrated embodiment, the quench vessel 64 provides counter-current direct contact between the gas feed 66 and water sprayed from one or more nozzles 68 positioned proximal to the top of the vessel. The quench vessel 64 can include a series of stacked trays 70 that facilitate direct contact between the rising gas and the falling liquid cooling medium. Alternatively, the quench vessel 64 can include a packed bed (not shown) to facilitate gas/liquid contact. As with the pipe 54, the quench vessel 64 can optionally also include a cooling water jacket 72 that receives a cooling water feed 74 and produces a cooling water effluent 76 such that the quench vessel simultaneously provides both direct and indirect contact with a cooling medium. Liquid cooling medium is withdrawn from the quench vessel 64 as liquid effluent 78 and gas exits the top of the vessel as gaseous effluent 80. As shown, the quench vessel 64 may further include a gas demister 82 proximal to the top of the vessel to reduce liquid entrainment within the gas exiting the vessel.

The quench system 50 can also include one or more heat exchangers for additional indirect heat exchange with the gas, such as heat exchanger 84, which can be included at any location within the system, such as upstream of the pipe 54, between the pipe and the quench vessel 64, within the quench vessel, and/or downstream of the quench vessel. The heat exchanger can be of any type and configuration suitable for gas to liquid heat exchange, including shell-and-tube heat exchangers and plate heat exchangers.

As shown, the gas leaving the quench system 50 is fed to a vacuum pump 86 where the gas is compressed and discharged in order to reduce the pressure within the upstream reactor. The quench system 50 can also include one or more additional gas-liquid separators (not shown), such as flash drums, knock-out drums, knock-out pots, compressor suction drums, and the like, at any suitable location upstream of the vacuum pump 86.

As shown, optionally, a recirculation line 90 from the vacuum pump 86 outlet to the vacuum pump inlet can be used to provide at least partial recirculation of the effluent from the vacuum pump to the pump inlet. A flow regulator (not shown) can be used to adjust the flow in the recirculation line 90 in order to handle disruptions in feed flow to the vacuum pump and still maintain desired reactor pressure.

### EXPERIMENTAL

Model-based sensitivity studies were performed using Aspen Plus to assess the energy savings benefit of the proposed concept against conventional steam ejectors based on the following basis and assumptions:
Reduction Gas (suction fluid) temperature: 600° C.;
Molecular weight of suction fluid: 25.5 Kg/Kmol
Quench water consumption calculated at adiabatic conditions;
Quench feed water at 40° C.;
Gas exit conditions at saturation;
Isentropic efficiency of vacuum pump taken as 75%;
Electric drive train efficiency is 95% and Grid efficiency 37.5%;
Motive steam pressure at 10-12 Bar and temperatures 200-300° C.; and
Motive steam energy requirements estimated based on steam-jet ejector charts available from open literature references.

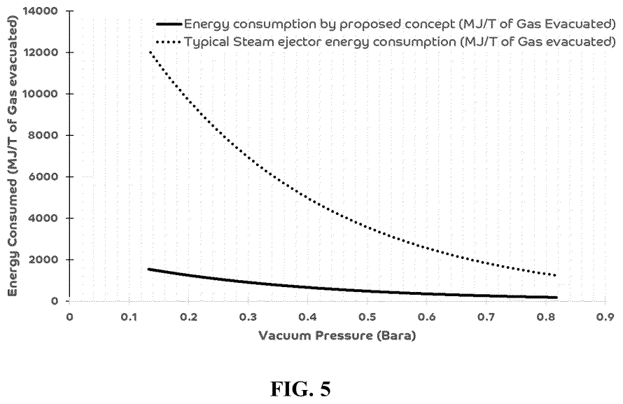

Based on the above assumptions, a graph of energy consumption (MJ/T of reduction gas evacuated) is modeled and shown as FIG. 5, showing the comparison between the proposed quench system/vacuum pump modeled using an equilibrium flash separation approach versus use of a conventional steam jet ejector. From the graph, it is clear that the proposed system consumes much less energy per ton of reduction gas evacuated than a conventional steam ejection system (where significant steam energy contained as latent heat is lost). The gap is particularly significant at lower vacuum pressures. The CO₂ emissions associated with energy are directly translatable based on CO₂ emission factor/MJ of energy consumed (depending on the fuel composition & calorific value). This evaluation indicates that the proposed system can offer a competitive advantage to improve energy efficiency and reduce carbon emissions for reduction gas evacuation and vacuum generation for high temperature operation applications processes, such as dehydrogenation of hydrocarbons.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

1. A method of evacuating a high temperature reactor requiring vacuum pressure during one or more steps of the reaction process, comprising:
a) directing a reduction gas stream from the reactor to a quench system in fluid communication with the reactor, the quench system comprising one or more enclosures wherein the reduction gas stream directly contacts a liquid cooling medium, wherein the liquid cooling medium is water;
b) withdrawing a cooled gas stream from the quench system; and
c) directing the cooled gas stream to a vacuum pump in fluid communication with the quench system and the reactor to reduce the pressure within the reactor to a vacuum pressure.

2. The method of claim 1, wherein the quench system comprises:
i) a pipe in fluid communication with the reactor and equipped with one or more nozzles positioned to spray cooling medium into direct contact with the reduction gas stream within the pipe; and/or
ii) a vessel in fluid communication with the reactor and equipped with one or more nozzles positioned to spray cooling medium into direct contact with the reduction gas stream within the vessel, the vessel comprising a gas exit for gaseous effluent and a cooling medium exit for liquid cooling medium effluent; and/or
iii) a liquid-cooled heat exchanger in fluid communication with the reactor; and/or
iv) any combination of two or more of i), ii), and iii).

3. The method of claim 1, further comprising venting the cooled gas stream from the vacuum pump to atmosphere.

4. The method of claim 1, wherein the vacuum pump is a dry vacuum pump or a liquid ring vacuum pump.

5. The method of claim 1, wherein the temperature of the cooled gas stream directed to the vacuum pump is about 150° C. or less.

6. The method of claim 1, wherein the reactor is evacuated to a vacuum pressure of about 0.6 bara or less.

7. The method of claim 1, wherein the reduction gas stream from the reactor directed to the quench system has a temperature of about 400° C. to about 800° C.

8. The method of claim 1, wherein the reactor is a dehydrogenation reactor.

9. A system for evacuating a high temperature reactor requiring vacuum pressure during one or more steps of the reaction process, comprising:
a) a high temperature reactor;
b) a quench system in fluid communication with the reactor and positioned to receive a reduction gas stream from the reactor, the quench system comprising one or more enclosures wherein the reduction gas stream directly contacts a liquid cooling medium, wherein the liquid cooling medium is water;
c) a source of liquid cooling medium in fluid communication with the quench system; and
d) a vacuum pump in fluid communication with the quench system and the reactor and positioned to receive a cooled gas stream from the quench system, the vacuum pump adapted to reduce the pressure within the reactor to a vacuum pressure.

10. The system of claim 9, wherein the quench system comprises:
i) a pipe in fluid communication with the reactor and equipped with one or more nozzles positioned to spray cooling medium into direct contact with the reduction gas stream within the pipe; and/or
ii) a vessel in fluid communication with the reactor and equipped with one or more nozzles positioned to spray cooling medium into direct contact with the reduction gas stream within the vessel, the vessel comprising a gas exit for gaseous effluent and a cooling medium exit for liquid cooling medium effluent; and/or
iii) a liquid-cooled heat exchanger in fluid communication with the reactor; and/or
iv) any combination of two or more of i), ii), and iii).

11. The system of claim 10, wherein the vessel is a vertical quench column comprising a demister proximal to a top of the column, a plurality of spray nozzles adapted for directing a cooling medium spray toward a bottom of the column, either packing or a plurality of trays between the spray nozzles and the bottom of the column, and a gas inlet proximal to the bottom of the column, wherein the gas exit is above the demister.

12. The system of claim 9, wherein the vacuum pump is vented to atmosphere for release of the cooled gas stream.

13. The system of claim 9, wherein the vacuum pump is a dry vacuum pump or a liquid ring vacuum pump.

14. The system of claim 9, wherein the vacuum pump is adapted for evacuating the reactor to a vacuum pressure of about 0.6 bara or less.

15. The system of claim 9, wherein the reactor is a dehydrogenation reactor.
